(19) United States
(12) Patent Application Publication
(10) Pub. No.: US 2008/0177250 A1
(43) Pub. Date: Jul. 24, 2008

(54) NESTABLE STERILITY-PROTECTING CAPS FOR SEPARATED CONNECTORS

(76) Inventors: Michael W. Howlett, Salt Lake City, UT (US); James V. Mercer, West Jordan, UT (US)

Correspondence Address: WORKMAN NYDEGGER, 60 EAST SOUTH TEMPLE, 1000 EAGLE GATE TOWER, SALT LAKE CITY, UT, 84111, US

(21) Appl. No.: 12/014,388

(22) Filed: Jan. 15, 2008

Related U.S. Application Data
(60) Provisional application No. 60/880,541, filed on Jan. 16, 2007.

(51) Int. Cl. A61M 39/16

(52) U.S. Cl. 604/533

(57) ABSTRACT

A pair of nestable caps are disclosed, each of the caps being sized and shaped to provide a protective union about a separated medical connector. The pair comprises a male cap and a female cap, each of which is configured to be adjoined to a complimentary cap to form a nested pair. The nested pair is sealed until separated for use, thereby maintaining sterility of the internal surfaces of the nested pair. An absorbent pad may be impregnated with antiseptic agent and enclosed within the pair to further enhance resulting sterility of each cap as it is used about a medical connector. One of the caps can include a scrubbing chamber having a disposable liner, a pad, and an antiseptic agent. Further, a pliable tube containing a frangible capsule may be coupled to one of the caps to provide antiseptic solution internal to the caps.

### CROSS-REFERENCE TO RELATED APPLICATIONS

This application claims the benefit of U.S. Provisional Application No. 60/880,541, titled ANTISEPTIC PROTECTIVE CAP FOR MALE AND FEMALE SCREW-TOGETHER CONNECTORS, filed Jan. 16, 2007, which is incorporated herein by reference in its entirety.

### BACKGROUND

1. The Field of the Invention

This invention is generally related to caps for medical connectors and specifically related to caps used to protect the sterility of separated medical fluid-flow connectors.

2. The Relevant Technology

Catheter-related bloodstream infections are caused by bacteria/fungi in patients with intravascular catheters. These infections are an important cause of illness and excess medical costs. Approximately 80,000 catheter-related bloodstream infections occur in U.S. intensive care units annually. Additionally, such infections are associated with anywhere from 2,400 to 20,000 deaths per year.

Guidelines from the Centers for Disease Control and Prevention describe various ways to limit catheter-related bloodstream infections in hospital, outpatient and home care settings. The guidelines address issues such as hand hygiene, catheter site care and admixture preparation. However, despite these guidelines, catheter-related bloodstream infections continue to plague our healthcare system.

Impregnating catheters with various antimicrobial agents is one approach that has been implemented to prevent these infections. These catheters, however, have given less than satisfactory results. Additionally, some microbes have developed resistance to the various antimicrobial agents in the system.

In another system that is commercially available in Europe, a catheter hub containing an antiseptic chamber is filled with three percent iodinated alcohol. Though it has shown to be effective, the catheter hub is expensive and does not fare as well in a formal cost-benefit analysis.

Caps used for protecting sterility of medical connectors are well known in the medical art. Commonly, protective tip caps are applied to luer connectors of tubing, IV access devices, stopcocks and syringes. Many examples of such tip caps are commercially available. Most commercially available medical tubing sets are packaged with a cap in place to protect the tubing during handling. Caps for other types of connectors, including catheter injection ports, are less common, though also known in the medical capping art.

Caps commonly used to cover medical connectors include a cover that is open at one end, closed at the other end and includes a set of spiraling screw threads (for making a secure and sealed luer type connection) over an associated bared connector, such as an injection port. The inside of the closed end can be equipped with a plastic capsule that can be ruptured when the cover is affixed to the associated connector. Rupturing of the plastic capsule releases antiseptic agents stored in the capsule, thereby applying the antiseptic agents to accessible portions of the connector.

One of the many examples of medical connectors for which such caps are used are intravascular connectors associated with a central line. Commonly, a central line is used to intermittently administer medicament to a patient. For example, a central line, which communicates fluids with a patient's blood stream, may have one or more connectors associated therewith. Each of the central line connectors can be connected to other connectors, such as a connector associated with an IV bag. In such a situation, the medical connectors, such as luer lock connectors, are connected and disconnected at various times, and may remain disconnected for several minutes or hours. Medical connector caps are used to cover and protect the various medical connectors while the connectors are disconnected from one another. When the medical connectors are disconnected from each other, there are two connectors that can require covering by a cap. Therefore, it would be an advantage to have a single connector set that can be used to provide protection for both ends of a separated connection.

### BRIEF SUMMARY

One embodiment of the present invention comprises a nested pair of protective caps having a male cap and a female cap. Each cap has threads which correspond to connectors generally used in medical apparatuses and which are separated for access thereto. Such medical apparatuses may include, but are not limited to, IV tubing sets, needleless injection sites or ports and vascular access devices. Each cap has threads which connect to a threaded end of an associated cap. As such, a pair of protective caps may be nested together to provide a pair of caps which, before separation, maintain sterile internal surfaces. These caps may subsequently be taken apart and applied to protect both ends of a separated medical connection.

Each protective cap encloses an associated medical apparatus connector and prevents touch contamination that may lead to microbial contamination, colonization and infection while the medical apparatus connectors are unattached. Further, in some embodiments, the protective caps can contain antiseptic agents that kill microorganisms such as bacteria, viruses and fungi that may colonize and lead to body-wide infection (e.g. IV catheter related blood stream infections). In such cases, the protective cap has an absorbent material for applying an antiseptic to an attached medical apparatus connector. The absorbent material also may provide a friction scrub while the protective cap is being connected. Such scrubbing improves microbial kill.

The “nesting” geometry of the pair of caps provides both caps as a single unit, sealed against contamination of connecting parts until the nested pair is separated. Generally, the nested pair is connected by the same thread geometries which provide for connecting to associated medical connecting apparatuses. When nested before use, the individual female and male caps are screwed together to form a seal to insure the sterility of the internal surfaces of each cap is maintained. For this reason, the nested pair unit or device does not require further sterilization before use as the unit is produced and delivered as an inherently self-sealed sterile package.

For example, the male cap from the device may be generally used to cover the end of an IV tubing set that is disconnected from an IV catheter needleless injection site. Examples of needleless injection sites, sometimes referred to as ports, hubs and valves, include brands such as Clave (ICU Medical), SmartSite (Cardinal) and Q-Site (Becton Dickenson). The female cap, from the nested pair, may then be used to protect the needleless injection site, itself. Importantly, once the cap has been applied, the medical apparatus connector need not be re-disinfected (e.g. treated with an alcohol swab) prior to each reconnection as it will be kept in an uncontaminated state while under the protective cap.

In an exemplary embodiment, the nested pair of pre-sterilized caps is packaged to protect against contamination by a seal covering about the junction of the two caps. Exemplary embodiments of the nested pair having a female portion, also contain a scrubbing material in a closed end of the female portion. The material is impregnated with an antiseptic agent. The male portion screws into corresponding threads of the female portion. To assure sterility and prevent fluid loss, the abutting edges of the male and female portions form a seal impermeable to passage of fluid and microbes when tightly affixed together. Alternatively, an O-ring may be affixed about a portion containing internal threads of the male cap. Such a seal reduces or prevents evaporative loss of antiseptic. A second, wrap-around seal may also or otherwise be used to provide additional protection for transport and storage. Unscrewing, to separate the two portions, breaks each such seal and scrubs the male portion. As indicated, the material disposed in the female portion scrubs the male connector of the medical apparatus being capped. This, in addition to prevention of contamination, thereby eliminates need for swabbing (e.g. by alcohol).

Other exemplary embodiments provide nested and sealed male and female portions and an internal antiseptic source as described above. However, to provide fresh antiseptic fluid within the nested pair prior to separation, a closed tube containing a frangible reservoir applicator is aseptically and sealingly attached to the unthreaded end of the female portion. The frangible reservoir provides an antiseptic-conveying open neck which passes into the female/male cap assembly. Internal to the tube is a capsule, containing the reservoir, which is manually crushable within the tube. At the female cap adjoining end of the tube, a pad of absorbent material is disposed to controllably communicate fluid resident in the reservoir into the interior of the nested pair. Thus, a pathway for release of antiseptic into the material and nested cap assembly is provided. Note that the act of twisting the male and female portions occurs naturally when the caps are parted. Such twisting causes the pad of absorbent material to swab internally exposed parts of the nested pair.

Yet other exemplary embodiments provide nested and sealed male and female portions as described above. In addition, at least one of the caps includes a scrubbing chamber disposed in an end thereof. The scrubbing chamber includes a cavity within one of the caps, a disposable liner removably position within the cavity, and a pad secured within the liner. The pad can be saturated or impregnated with an antiseptic agent. The scrubbing chamber can be used to clean and remove hazardous material from a medical connector prior to coupling another medical connector or one of the caps thereto.

Additional features and advantages of the present invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

FIG. 1 is a side elevation of an attached pair of medical caps made according to the instant invention;

FIG. 1A is a first end perspective view of the caps of FIG. 1;

FIG. 1B is a second end perspective view of the caps of FIG. 1;

FIG. 2 is an exploded perspective view of the medical caps of FIG. 1;

FIG. 3 is a perspective of a single cap portion of the attached pair seen in FIG. 2, with internal threads seen therein;

FIG. 4 is a perspective of the cap seen in FIG. 3 with an associated medical connector seen to be about to be connected thereto;

FIG. 5 is a perspective of a cap, which is complimentary to the cap of FIG. 3, and a luer lock connector to which the complimentary cap may be affixed;

FIG. 6 is a side elevation of an attached pair of medical caps, similar to the caps seen in FIG. 1, but having an “O” ring disposed about connecting edges of the caps;

FIG. 7 is an exploded view of the cap assembly seen in FIG. 6;

FIG. 7A is an end perspective view of one of the caps seen in FIG. 7 with a sealing mechanism disposed thereon;

FIG. 7B is an end perspective view of the other cap seen in FIG. 7 with a sealing mechanism disposed thereon;

FIG. 8A is a side elevation of the interconnected cap assembly seen in FIG. 6 with a planar or foil seal partially displaced about connecting edges of the cap assembly;

FIG. 8B is a side elevation of the interconnected cap assembly seen in FIG. 8A with the planar or foil seal fully in place;

FIG. 9A is a side elevation of the cap portion which is seen in FIG. 3 and an absorbent pad being disposed there above;

FIG. 9B is a side elevation of the cap portion and pad seen in FIG. 9A with the absorbent pad disposed within the cap portion;

FIG. 9C is a side elevation of the cap portion and pad as seen in FIG. 9B with a quantity of antiseptic material being dispensed into the cap and pad;

FIG. 9D is a horizontal perspective of the cap portion containing the pad as seen in FIGS. 9B and 9C affixed to an associated complimentary cap;

FIG. 10 is an exploded side elevation of a pair of complimentary caps, similar to the caps seen in FIG. 1, but having a tube portion which is sized and shaped to fit through a hole in the female portion of the nested pair;

FIG. 11 is a side elevation of the complimentary caps seen in FIG. 10 interconnected with the tube portion;

FIG. 12 is a side elevation of a pair of complimentary caps, similar to caps seen in FIG. 9D, with a second absorbent pad disposed therein;

FIG. 13 a perspective view of a pair of complimentary caps, similar to the caps seen in FIG. 1, but having an additional scrubbing device disposed in an end of one of the caps;

FIG. 14 is a cross-sectional perspective view of the pair of caps of FIG. 13.

### DETAILED DESCRIPTION

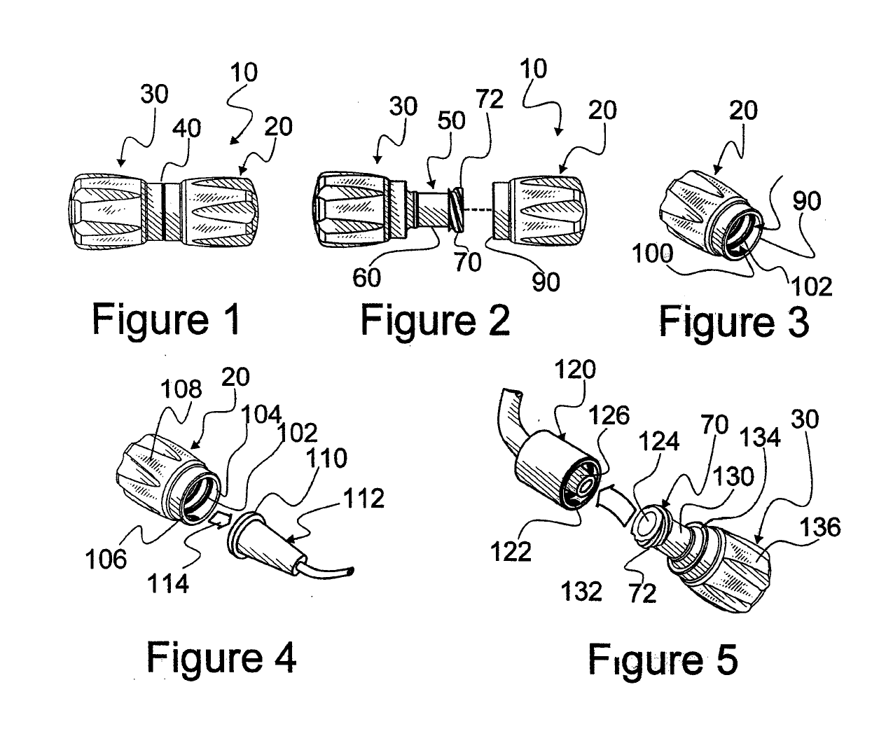

In this description, the term proximal is used to indicate that segment of a device normally closest to an object of the sentence describing its position. The term distal refers to an oppositely disposed position. Reference is now made to the embodiments illustrated in FIGS. 1-12 wherein like numerals are used to designate like parts throughout. Parts designated by numerals with primes are similar but not exactly the same as parts designated by numerals alone

Seen in FIGS. 1-1B, is a unit or assembly 10 of a pair of separable caps 20 and 30 securely, but releasably affixed one to the other across a common interface 40. To serve a meaningful purpose, internal parts and surfaces of assembly 10 must be sterile and, perhaps more important, be able to prevent contamination of a connector with which each cap becomes associated.

Caps 20 and 30 are seen apart in FIG. 2, wherein cap 30 is seen to have an insertable or male section 50. Section 50 has an elongated portion 60 which ends at an exteriorly disposed threaded segment 70. Threaded segment 70 comprises threads, generally numbered 72, which are sized and shaped to be inserted and joined by threading into cap 20.

Cap 20, which is better seen in FIG. 3, has a closed, hollow interior 80 which opens outwardly at a proximal end 90 to expose an interiorly disposed threaded segment 100. Threads 102 are of a size and pitch to complimentarily engage threads 72 for a screw or push on tight fit with cap 30.

As illustrated in FIG. 4, cap 20 has an interior surface 104, an opening edge 106 and an exterior surface 108, opening edge 106 being a common link between interior surface 104 and exterior surface 108. Further, threads 102 also have a size and pitch to engage a threadable segment 110 of a female connector, such as by example, female luer connector 112. Such connectors are generally and commonly used as catheter and other fluid tight, protective connectors in medical applications. As seen in FIG. 4, cap 20 provides a protective cover for connector 112 when encased about connector 112 (displaced in direction of arrow 114) where upon threadable segment 110 engages and is drawn into a secure, but releasable connection with threads 102 of cap 20.

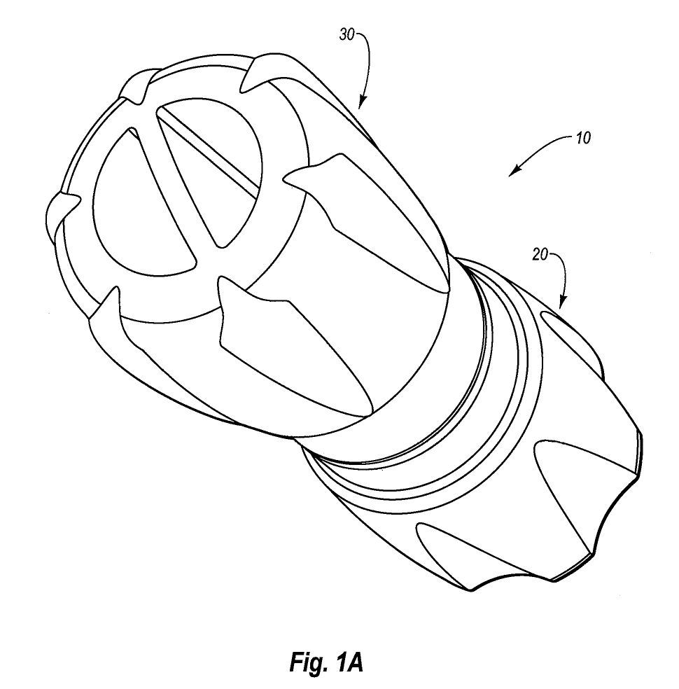

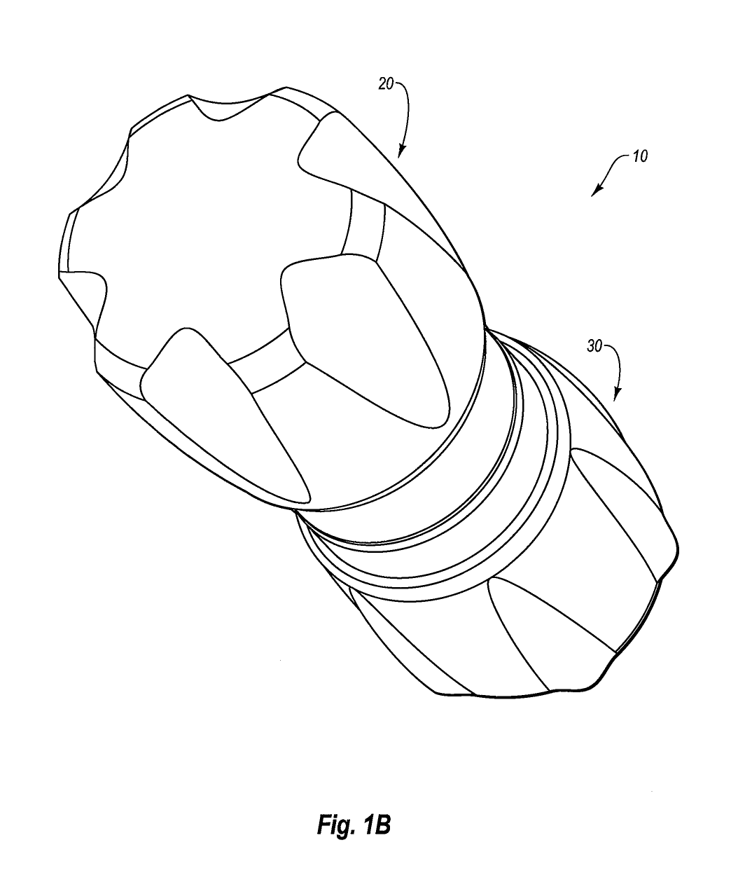

Likewise, as seen in FIG. 5, threads 72 of cap 30 are of a size and pitch to engage threads 122 of a male luer-lock connector 120. Male connectors, of which connector 120 is but a single example, are also commonly used to protect fluid connections in the medical art. Note that cap 30 has a medially disposed, elongated hole 124, into which a frustoconical cone shaped luer 126 of connector 120 may be facilely and securely inserted when cap 30 is displaced in direction of arrow 128 to engage connector 120.

Cap 30 also has a surface 130 which continues through to a circular edge 132. Further, distally displaced from circular, edge 132, surface 130 abruptly ends at a circular ring shaped edge 134 which is therefrom joined to an outside surface 136. It may be noted that opening edge 106 (see FIG. 5) and ring shaped edge 134 combine to form common interface 40 (see FIG. 1) when cap 20 is affixed to cap 30 to construct assembly 10. It should also be noted that surfaces of assembly 10 which contact internal surfaces of a connector, such as connector 112 or connector 120, should be sufficiently sterile to not contaminate the inner surfaces thereof.

For this reason, internal portions and associated edges of caps 20 and 30 should be pre-sterilized and so maintained until use. Caps 20 and 30 may be injection molded using polypropylene or other material which can be sterilized and which is impervious to contaminating agents while cap 20 is fully nested with cap 30, before being opened for use. Caps 20 and 30 can also be impregnated or coated with an antimicrobial substance. As an example, each cap 20 and cap 30 may be individually sterilized by ethylene oxide (ETO) before final assembly and aseptically paired, or assembly 10 may be finally consolidated as a single unit and then sterilized, such as by radiation (e.g. gamma). Even so, assembly 10 should be kept intact until time for use, with internal surfaces of nested parts 20 and 30 remaining clean and sterile until assembly 10 is opened for use.

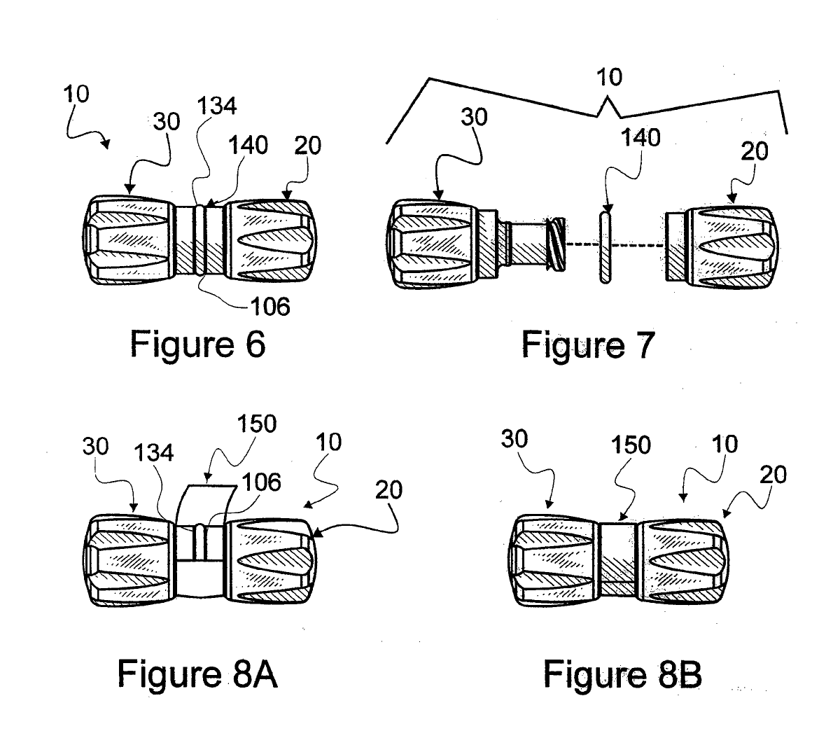

Reference is now made to FIGS. 6 through 7B wherein a seal, such as an “O” ring, is disposed between surfaces 106 and 134 to provide yet another barrier against internal surface contamination of caps 20 and 30. As seen in FIG. 6, an “O” ring 140 is disposed between surfaces 106 and 134 to provide a seal thereby. While “O” ring 140 can be displaced from caps 20 and 30 as illustrated in FIG. 7, it is anticipated that “O” ring 140 can be adapted to remain affixed to one of caps 20 and 30. For example, as illustrated in FIG. 7A, “O” ring 140 can remain positioned adjacent surface 134 on cap 30 when caps 20 and 30 are disconnected from one another, rather than being separated when cap 30 is displaced from cap 20, as seen in FIG. 7.

Alternatively, “O” ring 140 can be associated with cap 20, as seen in FIG. 7B. In particular, opening edge 106 of cap 20 can have an annular groove 116 for receiving “O” ring 140 therein. Annular groove 116 can be sized and shaped such that “O” ring 140 sealingly engages cap 30 or a medical connector when cap 20 is coupled thereto. It will be appreciated that annular groove 116 can be disposed in opening edge 106 toward the exterior of cap 20 as illustrated in FIG. 7B, or annular groove 116 can be disposed in opening edge 106 towards the interior of cap 20. In some exemplary embodiments, opening edge 106 of cap 20 does not have annular groove 116 therein. In such embodiments, “O” ring 140 can be mounted directly to opening edge 106. “O” ring 140 can be mounted on or to caps 20 or 30 in any suitable manner, including with the use of an adhesive, such as glue, a mechanical fastener, or a friction fitting.

As noted elsewhere herein, a sealing mechanism, such as “O” ring 140, can be used to limit or prevent evaporation or loss of an antiseptic agent disposed within caps 20 and 30 when caps 20 and 30 are coupled together. Additionally, a sealing mechanism, such as “O” ring 140, can also limit or prevent evaporation or loss of an antiseptic agent disposed within caps 20 and 30 when caps 20 and 30 are coupled to separated medical connectors. Further, a sealing mechanism, such as “O” ring 140, can also limit or prevent microbial ingress within caps 20 and 30 when coupled to each other, or within caps 20 and 30 when caps 20 and 30 are coupled to separated medical connectors. Moreover, a sealing mechanism, such as “O” ring 140, can be adapted to maintain an antiseptic agent within caps 20 and 30 when caps 20 and 30 are either coupled to one another or to separated medical connectors for a predetermined amount of time.

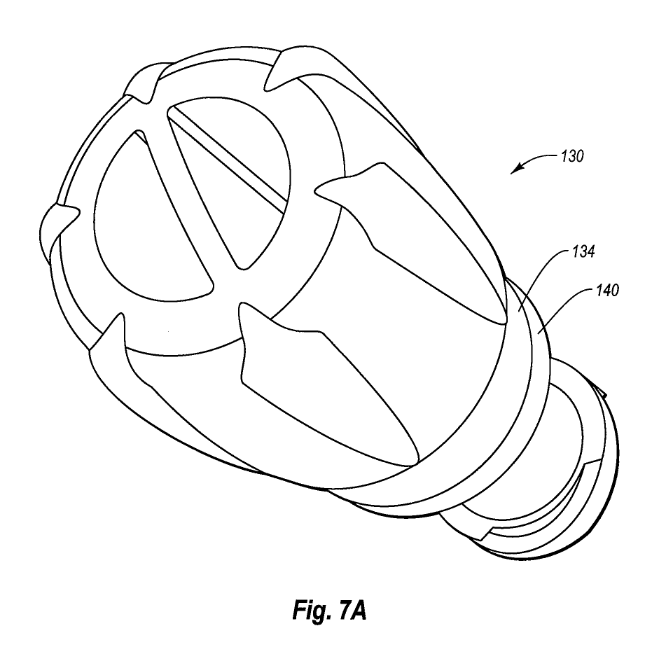

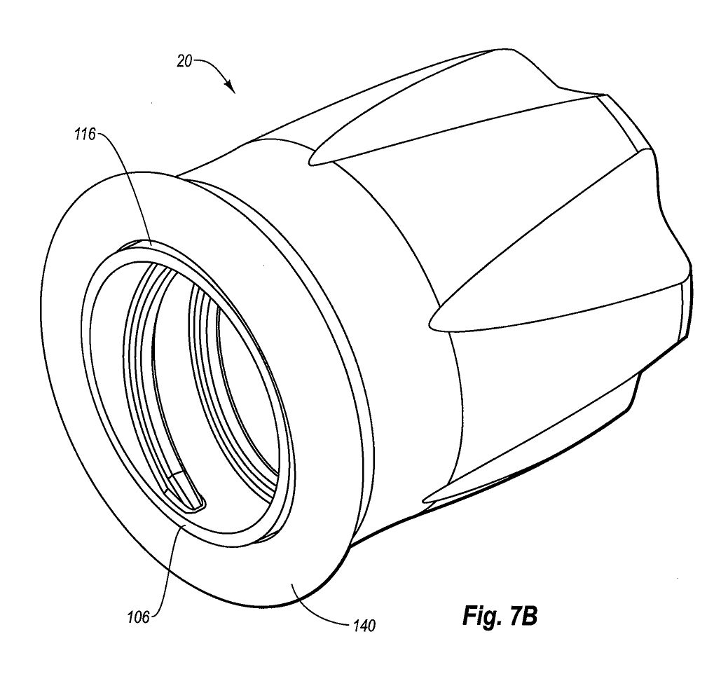

Further safety in sealing against internal surface contamination may be provided by a sealing tape, such as tape 150 seen in FIG. 8A. Tape 150 is disposed to fully cover exposed edges of surfaces 106 and 134. Tape 150 may, for example, be of an impervious pliable material, such as a metallized-surface mylar. As seen in FIG. 8B, tape 150 is wrapped about surfaces 106 and 134 to provide a secure seal. It is preferred that tape 150 frangibly divides when cap 20 is separated from cap 30.

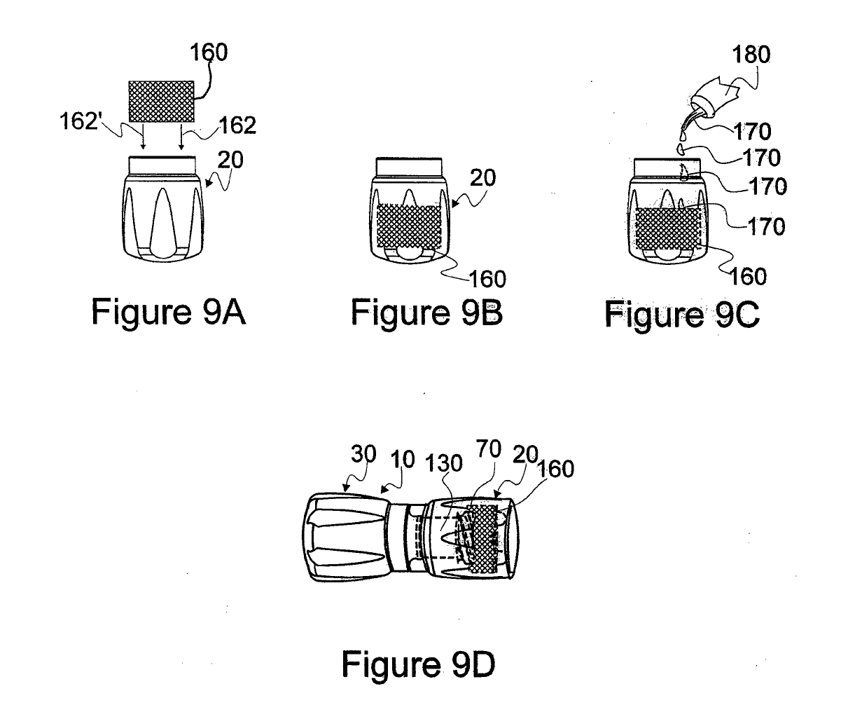

When capping disconnected medical connectors, it is prudent to do more than just cover those connectors with caps. For this reason, an absorbent pad, such as pad 160, seen in FIG. 9A, may be displaced into cap 20 as indicated by arrows 162 and 162′. Pad 160 is seen disposed in cap 20 in FIG. 9B. An antiseptic 170 can also be disposed within cap 20. Antiseptic 170 can be in liquid or solid form. For example, alcohol or another stable liquid antiseptic may be added as from a container 180 to saturate pad 160 to a predetermined level. Note that once assembly 10 is fully assembled, pad 160 will substantially remain at the predetermined saturated level due to the exterior seals provided for assembly 10 as described above. Alternatively, or additionally, pad 160 may be impregnated with a dry antiseptic, such as chlorhexidine gluconate.

Further note that, once cap 30 is securely affixed to cap 20, as seen in FIG. 9D, pad 160 is disposed to contact at least circular edge 132 (see also FIG. 5). (In FIG. 9D, parts of cap 30 which are internal to assembly 10 are seen with hidden or dashed lines.) Such contact provides a wiping action preferred to make contact with a surface before contact is made with an associated connector. Note also that residual antiseptic on associated internal surfaces of cap 30 may be transferred to related parts of the associated connector for cleaning purposes.

Pad 160 can be formed of a deformable, resilient material such that when cap 30 is coupled to cap 20, elongated portion 60 can compress pad 160 within cap 20. Further, pad 160 can expand to its original shape when cap 30 is removed from cap 20. Similarly, pad 160 can be compressed within cap 20 when cap 20 is coupled to a medical connector, such as medical connector 112. Pad 160 can also be formed such that when a medical connector is coupled to cap 20, pad 160 is deformed such that pad 160 extends around the threads of the medical connector. For example, pad 160 can be formed such that as cap 20 is twisted onto medical connector 112, pad 160 deforms around threads 110, thereby scrubbing threads 110.

Similar to pad 160 and antiseptic 170 disposed within cap 20, and as described elsewhere herein, cap 30 may also have a pad and/or an antiseptic disposed therein. For example, a pad and/or antiseptic may be disposed within elongate hole 124 of cap 30 (FIG. 5). Once assembly 10 is fully assembled, a pad disposed within cap 30 which has been saturated with an antiseptic will substantially remain at the predetermined saturation level due to the exterior seals for assembly 10 as described above. Once caps 20 and 30 are disconnected from each other and connected to individual medical connectors, the pad and/or antiseptic disposed within cap 30 may be transferred to related parts of the associated connector for cleaning purposes.

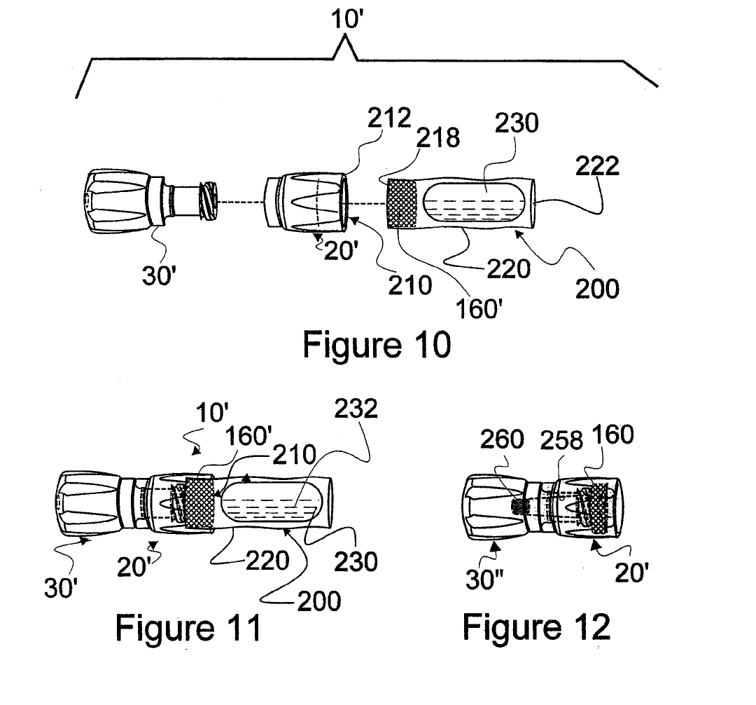

Under some conditions, it may be preferable to retain an antiseptic solution within a container prior to separating the nested caps. Reference is now made to FIGS. 10-11 wherein an assembly 10′ is seen to comprise a first cap 30′, a second cap 20′ and an antiseptic containing vessel 200.

As seen in FIG. 10, geometric features of cap 20′ are similar to features of cap 20 except for a through hole 210 in end 212, providing access into cap 20′. Rather than an absorbent pad 160 (see FIG. 9D), an absorbent pad 160′ is resident at an open end 218 of a hollow pliable tube 220. Tube 220 is closed at an opposite end 222. Also, retained in tube 220 by pad 160′ and closed end 222 is a frangible capsule 230 which is filled with a predetermined amount of antiseptic solution.

Note that tube 220 is sized and shaped and of a material to fill and be adhesively and scalingly affixed to cap 20′ to provide a seal about hole 210. A commercially available frangible reservoir tube such as the ChlorPrep Sepp Applicator provided by Enturia may be used for vessel 200.

As seen in FIG. 11, tube 220 is displaced into hole 210 to be adhesively affixed thereat. Resultingly, vessel 200 becomes an integral part of assembly 10′. Prior to separating cap 30′ from cap 20′, capsule 230 is manually crushed to cause fluid 232 contained therein to be released into the void of parts of assembly 10′ through pad 160′.

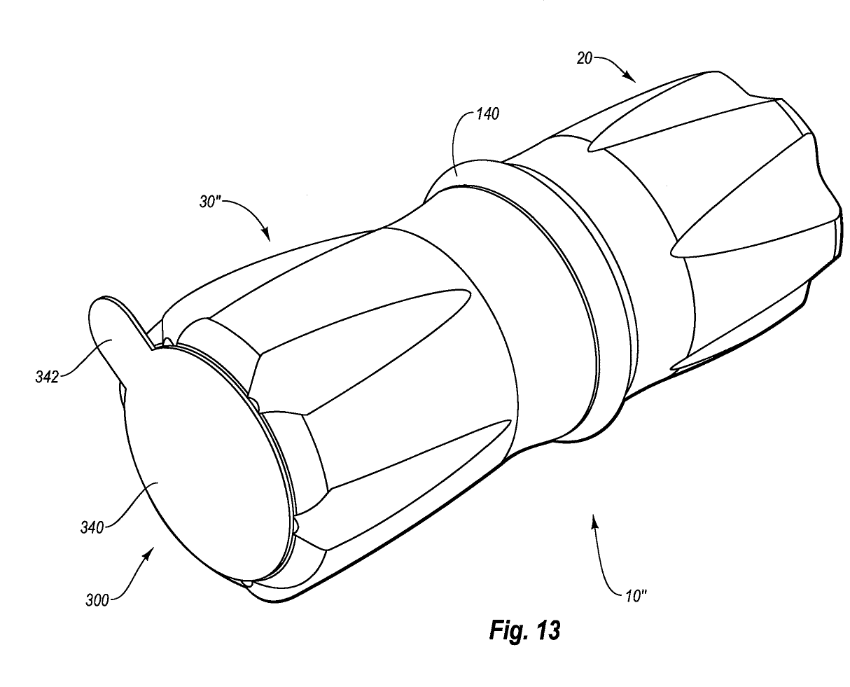

As mentioned for tube 230, it is common for a cap, such as cap 30 or 30′ to be used to protect a male luer connector. In such a case, a cap 30″ seen in FIG. 12 is seen to contain a hollow frustoconically shaped internal connector surface 258. To further cleanse a male luer connector inserted therein, a second antiseptic filled pad is disposed at the deepest point thereof for contact with the end of the male connector.

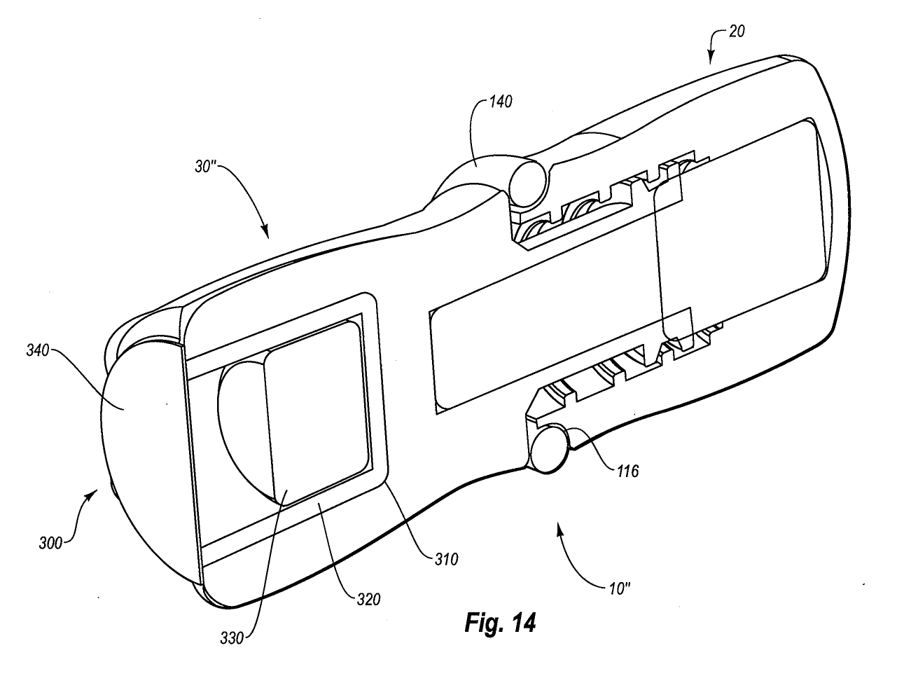

With attention to FIGS. 13 and 14, another exemplary embodiment of a nestable pair of caps 10″ is illustrated having a female cap 20 and a male cap 30″, similar to the caps previously described herein. Disposed between caps 20 and 30″ can be a sealing mechanism 140 as described above. Additionally, one or both of caps 20 and 30″ can have a scrubbing chamber 300 disposed within an end thereof. Scrubbing chamber 300 can be adapted to clean and disinfect a medical connector prior to connecting the medical connector to another medical connector or one of caps 20 and 30″.

In the illustrated embodiment, scrubbing chamber 300 is disposed within cap 30″, however, it will be appreciated that scrubbing chamber 300 can be disposed within the end of cap 20. Scrubbing chamber 300 comprises a cavity 310 within the end of cap 30″. Positioned within cavity 310 is a liner 320. Liner 320 can be removably secured within cavity 310 such that liner 320 can be securely held in place during use of cap 30″ and/or scrubbing chamber 300, and removed after use of scrubbing chamber 300. Liner 320 can also be adapted to be removed from cavity 310 prior to use of scrubbing chamber 300, such that scrubbing chamber 300 can be used independently of caps 20 or 30″. The removability of liner 320 allows scrubbing chamber 300 to be used to remove potentially hazardous material, such as blood, body fluids, and the like, off of a medical connector and then discard the contents of scrubbing chamber 300 so that the hazardous material does not remain near the medical connector when the cap is coupled thereto.

Disposed within liner 320 is pad 330. Similar to pad 160 described above, pad 330 can be formed of a deformable material capable of deforming such that pad 330 extends around the opening and threads of a medical connector. Pad 330 can also be formed of an abrasive material that can break up hardened materials that have collected on a medical connector. Additionally, pad 330 can be formed of a material that can be impregnated or saturated with an antiseptic agent, such as alcohol or chlorhexidine gluconate.

Scrubbing chamber 300 can also include a removable cover 340 for enclosing liner 320, pad 330, and any antiseptic agent within cavity 310. As illustrated in FIG. 14, cover 340 extends across the opening to cavity 310 and is secured to cap 30″. Cover 340 can be secured to cap 30″ is any manner that sealingly maintains the contents of cavity 310 therein and that allows for ready removal of cover 340 when scrubbing chamber 300 is to be used. Cover 340 can be formed of any suitable material, such as foil, plastic, and the like. Additionally, cover 340 can also include a tab 342 that facilitates quick and convenient removal of cover 340 when scrubbing chamber 300 is to be used.

The caps described herein can be formed of, or coated with various colored materials or coatings. In one exemplary embodiment, caps 20 and 30 comprise a single color. Alternatively, each of caps 20 and 30 can be a separate color. Coloring caps 20 and 30 can provide various advantages, such as ready identification of the type of cap, ready matching of a particularly colored cap with a particular type of medical connector, and the like.

The inventions disclosed herein may be embodied in other specific forms without departing from the spirit or essential characteristics thereof. The present embodiments are therefore to be considered in all respects as illustrative and not restrictive. The scope of the invention is, therefore, indicated by the appended claims rather than by the foregoing description. All changes which come within the meaning and range of equivalency of the claims are therefore intended to be embraced within their scope.

What is claimed is:

1. A nestable pair of caps for use in providing a protective union about a separated medical connector, the nestable pair comprising:
a male cap comprising a connecting geometry whereby the male cap may be selectively coupled to a female cap or a separated female medical connector;
a female cap comprising a connecting geometry whereby the female cap may be selectively coupled to a male cap or a separated male medical connector; and
each of the male and female caps comprises an internal surface, an external surface, and an interface, the interfaces of the male and female caps provide a seal when adjoined together and which aseptically partition the internal and external surfaces to insure perpetuated sterility of the internal surfaces.

2. A nestable pair of caps according to claim 1, wherein the seal further comprises an “O” ring.

3. A nestable pair of caps according to claim 1, wherein the seal further comprises a sealing tape disposed about the interfaces of the male and female caps.

4. A method according to claim 1, wherein the female cap comprises an internal well adapted to receive a pad therein.

5. A method according to claim 4, wherein the well of the female cap is adapted to receive an antiseptic agent therein.

6. A method for protecting separated ends of medical connectors, the method comprising the steps of:
providing a pair of caps, each of the caps sized and shaped to provide a protective union about a separated medical connector and to be selectively joined together, the pair comprising:
a male cap having a threaded projecting portion with a hollow interior portion, the male cap being adapted for selective coupling to a complimentary female cap or a separated female medical connector; and
a female cap having a threaded interior portion, the female cap being adapted for selective coupling to a complimentary male cap or a separated male medical connector by receiving at least a portion of the male cap or separated male medical connector within the threaded interior portion thereof;
adjoining the male cap and the female cap together to form a nested pair;
processing the pair to assure internal surfaces are sterile;
separating a pair of medical connectors, exposing an unattached female connector and an unattached male connector;
twisting the nested pair apart to provide the male cap and female cap;
coupling the female cap to the male connector; and
coupling the male cap to the female connector.

7. A method according to claim 6, wherein at least one of the male and female caps further comprises a scrubbing chamber, the scrubbing chamber having a removable liner positioned within an end of the male or female cap, a pad disposed within the liner, and a cover that sealingly encloses the interior the liner.

8. A method according to claim 7, wherein prior to twisting the nested pair apart, the cover of the scrubbing chamber is removed to expose the pad, the pad then being used to clean at least one of the separated medical connectors, and the liner and pad then being removed from the cap.

9. A method according to claim 6, wherein coupling of the female cap to the male connector comprises swabbing a portion of the male connector.

10. A method according to claim 6, wherein coupling of the female cap to the male connector and the male cap to the female connector comprises applying an antiseptic agent to the male connector and the female connector.

11. A nestable pair of caps for use in protecting separated medical connectors, comprising:
a first cap having an exterior surface and an interior surface, the exterior surface having an opening therein for access to the interior surface, the interior surface having a threaded portion; and
a second cap having an exterior surface and a frustoconical portion extending from the exterior surface, an outer surface of the frustoconical portion having a threaded portion adapted to engage the threaded interior portion of the first cap to enable the second cap to be selectively secured to and at least partially nested within the first cap.

12. A nestable pair of caps according to claim 11, wherein the female cap comprises an internal well adapted to receive an absorbent pad therein.

13. A nestable pair of caps according to claim 12, wherein the absorbent pad is adapted to be impregnated with an antiseptic agent.

14. A nestable pair of caps according to claim 12, wherein the male cap, the female cap, and the absorbent pad each comprises a geometry which causes the absorbent pad to be compressed within the female cap by a portion of the male cap when the male cap and female cap are joined together.

15. A nestable pair of caps for use in protecting separated medical connectors, comprising:
a first cap having an exterior surface defining an opening to a hollow, threaded interior portion, the hollow interior portion having an antiseptically saturated pad disposed therein, the hollow interior portion being adapted to receive a portion of a separated medical connector therein;
a second cap having an exterior surface, an elongated portion extending therefrom, the elongated portion having an elongated hole with an antiseptically saturated pad disposed therein, the elongated hole being adapted to receive a portion of a separated medical connector therein, and a scrubbing chamber disposed therein, the scrubbing chamber comprising a cavity within the second cap, a liner positioned within the cavity, a pad disposed within the liner, and a cover removably extending across the opening of the liner;
the first cap and the second cap being adapted for selective and sealing engagement with one another to prevent contamination of the hollow interior portion of the female cap and the elongated portion of the male cap prior to disengagement of the first cap and the second cap from one another.

16. A nestable pair of caps according to claim 15, wherein the female cap further comprises a depressible tube with a frangible capsule therein and an orifice through which contents of said tube communicate with the internal portions of the female cap.

17. A nestable pair of caps according to claim 16, wherein the tube contains a pad of absorbent material disposed at the orifice.

18. A nestable pair of caps according to claim 16, wherein the capsule further comprises an antiseptic solution which is communicated to the internal portions of the female cap by fracturing the capsule.

19. A nestable pair of caps according to claim 15, wherein the first cap is adapted to couplingly receive therein at least a portion of a separated male medical connector.

20. A nestable pair of caps according to claim 15, wherein the second cap is adapted to be coupled to a separated female medical connector, wherein the elongated portion of the second cap is at least partially received within the separated female medical connector and a portion of the female medical connector is received within the elongated hole of the second cap.

21. A nestable pair of caps for use in protecting separated medical connectors, comprising:
a female cap having a gripping portion and a hollow interior portion;
a male cap adapted for selective coupling to the female cap, the male cap having a gripping portion and a tubular portion adapted to project within the hollow interior portion of the female cap when the male cap is coupled to the female cap, the hollow interior portion and the tubular portion having cooperating geometries to facilitate secure engagement of the male cap and the female cap; and
a seal disposed between the female cap and the male cap, the seal being adapted to prevent contamination of the hollow interior portion of the female cap and the tubular portion of the male cap, the seal also being adapted to limit evaporation or loss of a antiseptic agent disposed within the pair of caps;
wherein the female cap and the male cap are adapted for selective disengagement from one another and individual engagement of separated medical connectors.

22. A method according to claim 21, wherein the female cap further comprises a depressible tube coupled thereto, the tube comprising a frangible capsule and an orifice through which contents of the tube communicate with the internal portions of the female cap.

23. A method according to claim 22, wherein the tube further comprises a pad of absorbent material disposed therein to obstruct the orifice.

24. A method according to claim 23, wherein the capsule further comprises an antiseptic solution which is released from containment by fracturing the capsule.

25. A method according to claim 24, wherein the tube is depressed to break the frangible capsule and release antiseptic solution through the pad and the orifice, thereby dispensing the antiseptic solution to the internal surfaces of the nested pair prior to twisting the nested pair apart.
